(19) United States
(12) Patent Application Publication
(10) Pub. No.: US 2003/0062425 A1
(43) Pub. Date: Apr. 3, 2003

(54) Air freshener device

(76) Inventor: Anthony Eugene Girone, Toms River, NJ (US)

Correspondence Address: Anthony Eugene Girone, 624 Broadway Blvd., Toms River, NJ, 08757, US

(21) Appl. No.: 10/228,327

(22) Filed: Aug. 26, 2002

Related U.S. Application Data
(60) Provisional application No. 60/314,550, filed on Aug. 24, 2001.

(51) Int. Cl. A61L 9/04

(52) U.S. Cl. 239/34; 428/905; 239/53

(57) ABSTRACT

air freshener device with the steps of: made of carpet, fake fur and felt, reusable, and fragrance can be changed by washing.

### CROSS REFERENCE TO RELATED APPLICATIONS

This application is based on provisional application serial No. 60/314,550, filed on Aug. 24,2001.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of air freshener device and more specifically to Kar-Pets.

### BRIEF SUMMARY OF THE INVENTION

The primary object of the invention is refillable.

Another object of the invention is attaches to many surfaces.

Another object of the invention is washable.

A further object of the invention is cute.

Yet another object of the invention is has many shapes.

Still yet another object of the invention is last for years.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed air freshener device comprising the steps of: made of carpet, fake fur and felt, reusable, and fragrance can be changed by washing.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

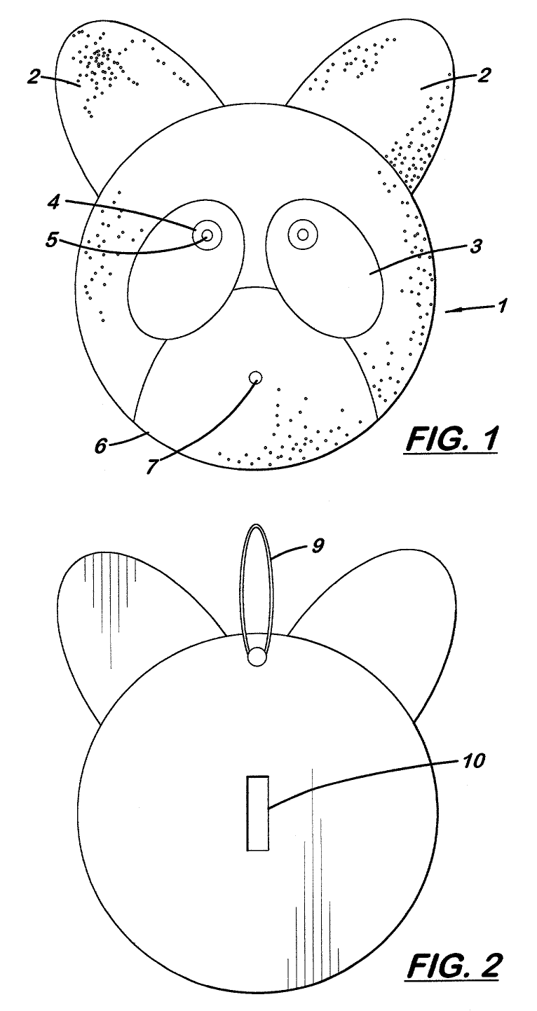

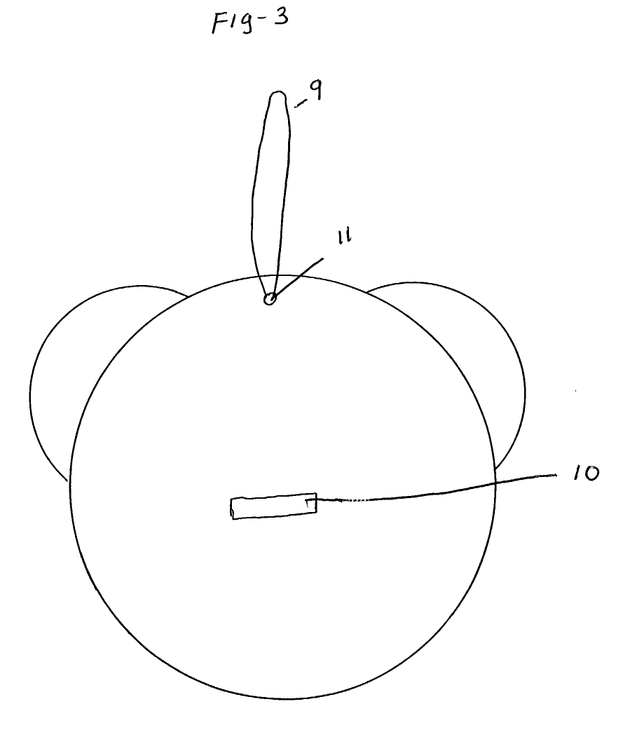

The main item of concern is 1 which is a pair of circles glued back to back to form a pouch in which a few drops of potpourri oil can be added to the inside with carpet absorbing scent. On this particular figure parts 2 are made by gluing two parts of felt back to back. Then glued to 1 to form the ears. Two parts 3 of felt are then glued in place to represent cheeks. Two parts 5 are glued to parts 4 and parts 4 are glued to part 3 to form eyes. Parts 6 made of fake fur is then glued to part 1 with part 7 glued to part 6. Refer to FIG. 3 part 10 is a magnet or velcro for attachment part 11 is a grommet for installing a suspension mechanism.

What is claimed is:

1. air freshener device comprising the steps of:
made of carpet, fake fur and felt;
reusable; and
fragrance can be changed by washing.
